(19) United States
(12) Patent Application Publication
(10) Pub. No.: US 2024/0099632 A1
(43) Pub. Date: Mar. 28, 2024

(54) NERVE STIMULATION PATTERNS AND DEVICE FOR DETECTABILITY OF NERVE FUNCTION AND HEALTH

(71) Applicant: CHECKPOINT SURGICAL, INC., Cleveland, OH (US)

(72) Inventors: Ben Cottrill, Cleveland, OH (US); Eric R. Walker, Berea, OH (US)

(21) Appl. No.: 18/038,458

(22) PCT Filed: Dec. 1, 2021
(86) PCT No.: PCT/US2021/061388

§ 371 Date: May 24, 2023

Related U.S. Application Data
(60) Provisional application No. 63/119,683, filed on Dec. 1, 2020.

(51) Int. Cl. A61B 5/294; A61B 5/00; A61B 5/388

(52) U.S. Cl. CPC A61B 5/294; A61B 5/388; A61B 5/6848

(57) ABSTRACT

An electrical stimulation system includes a stimulation device. The stimulation device may generate electrical stimulation in a stimulation pattern. Sensors may detect evoked potential. The electrical stimulation system may determine whether the evoked potential is a result of the electrical stimulation based at least in part on the stimulation pattern.

### CROSS-REFERENCE TO RELATED APPLICATION(S)

This application claims the benefit of U.S. Provisional Patent Application No. 63/119,683, entitled “Nerve Stimulation Patterns and Device for Detectability of Nerve Function and Health,” filed on Dec. 1, 2020, which is hereby incorporated by reference.

### FIELD OF THE INVENTION

The invention relates generally to nerve stimulation, and more particularly to systems and methods for stimulating innervated muscle and detecting evoked muscle contraction.

### BACKGROUND

Surgical procedures are not risk-free. Each patient's anatomy differs, requiring the surgeon to be ever vigilant to these differences so that the intended result is accomplished. The positioning of nerves and other tissues within a human or animal's body is one example of how internal anatomy differs from patient to patient. While these differences may be slight, if the surgeon fails to properly identify one or several nerves, the nerves may be compressed, stretched, or even severed during an operation. The negative effects of nerve damage can range from lack of feeling on that part of the body to loss of muscle control.

Traumatic injuries often require surgical repair. Determining the extent of muscle and nerve injury is not always possible using visual inspection. Use of an intra-operative stimulator enables accurate evaluation of the neuromuscular system in that area. This evaluation provides valuable knowledge to guide repair/reconstructive surgery following traumatic injury, and when performing a wide range of surgeries.

Intraoperative nerve integrity monitoring involves sonic and graphic display of electromyography activity from muscles of nerves at risk for surgical injury. In some traditional systems, specialized devices deliver an electronic stimulation to tissue and collect feedback elicited by electrical stimulation of the monitored nerve. This allows a surgeon to monitor nerves for potential harm. In such traditional systems, nerves may be verified as intact by periodically sending stimulation and examining the response via a monolithic piece of capital equipment requiring extensive technical support and expertise. A skilled operator monitors the device and interprets the evoked potentials picked up via electromyography monitoring, where recording and reviewing is triggered with stimulation pulses. Traditional systems may be prone to misuse, operator error, false alarms, and the like. Moreover, absolute thresholds for traditional systems are generally required. Such systems, likewise, typically suffer from inaccuracies due to electromyography noise, respiration induced noise, or errors caused through lost electrode connection.

It may be desirable to provide systems and methods for delivering stimulation output patterns that provide improved detection (e.g., automated detection, visual detection, etc.), improved automated detection, reduction in false readings, and the like. It may also be desirable to provide stimulation and detection via stimulation output patterns through a handheld stimulation device, through continuous intraoperative nerve integrity monitoring, or the like.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Described systems and methods provide for generation of stimulation by a stimulation device. The stimulation device generates pulses according to specified stimulation patterns. The stimulation pattern may include a plurality of pulses generated in groups with a time period separating successive groups.

An example electrical stimulation device includes a housing; an operative element extending from the housing, and a control circuitry in communication with the operative element, wherein the control circuit comprises a memory comprising instructions that when executed generate pulses of stimulus electrical stimulation that comprises (i) at least first and second groups with a group time between a first and a last pulse of each of the first and second groups, and (ii) a burst stimulation for a burst time between the first pulse of the first group and the first pulse of the second group, wherein the burst time is greater than the group time.

The electrical stimulation device described above may also include, singly or in any combination, (a) a burst stimulation with a frequency and amplitude to induce repeated slow-twitch contractions, (b) a user interface configured to modify a pattern of the burst stimulation used by control circuit based on input from the user interface, (c) a stimulation detection device configured to detect electrical stimulation to a target nerve, (d) a stimulation detection device to detect electrical stimulation to a target nerve that includes at least one of a return electrode, EMG sensor, a cuff, an endotracheal tube or a camera, (e) an indicator configured to confirm delivery of a predefined stimulation pattern to tissue, (f) a pulse counter configure to count a number of pulses applied, (g) a pulse counter configure to count a number of pulses applied that is also configured to turn off power when a predetermined number of pulses are detected by the pulse counter, (h) an evoked potential sensor configured to determine generation of an evoked potential from a nerve being stimulated, and/or (i) an evoked potential sensor configured to determine generation of an evoked potential from a nerve being stimulated that includes an accelerometer or an electrode. In some examples, the burst stimulation with may have a frequency of (a) 1.6 Hz with a stimulus electrical frequency is 16 Hz, (b) 2.7 Hz with a stimulus electrical frequency is 16 Hz (c) 3.2 Hz with a stimulus electrical frequency is 32 Hz, or (d) 4 Hz with a stimulus electrical frequency is 32 Hz.

An example electrical stimulation device includes a housing, an operative element extending from the housing, and a control circuitry in communication with the operative element, wherein the control circuit comprises a memory comprising instructions that when executed generate pulses of stimulus electrical stimulation in a first group, wherein the pulses in the first group each comprise different parameters. In some examples, the first group comprises three pulses wherein each of the pulses comprise different amplitudes. In some examples, the first group comprises a plurality of pulses wherein each pulse comprises a different pulse duration.

An example electrical stimulation device includes a housing, an operative element extending from the housing, and a control circuitry in communication with the operative element, wherein the control circuit generates pulses of electrical stimulation in a first group with a first group time between a first and a last pulse of the first group and a second group with a second group time between a first pulse and a last pulse and a burst stimulation for a burst time between the first pulse of the first group and the first pulse of the second group, wherein the burst time is greater than each of the first group time and the second group time. In some examples, the burst stimulation comprises a frequency and amplitude to induce repeated slow-twitch contractions. In some examples, the control circuit comprises a memory comprising third and fourth groups of pulses of electrical stimulation wherein stimulation parameters of the first and second groups are different from stimulation parameters of the third and fourth groups. In some examples, an evoked potential sensor is configured to determine generation of an evoked potential from a nerve being stimulated, wherein the evoked potential sensor changes the electrical stimulation from the first and second groups to the third and fourth groups.

The following description and the drawings disclose various illustrative aspects. Some improvements and novel aspects may be expressly identified, while others may be apparent from the description and drawings.

### BRIEF DESCRIPTION OF THE DRAWINGS

The accompanying drawings illustrate various systems, apparatuses, devices and methods, in which like reference characters refer to like parts throughout.

FIG. 1 illustrates a partial cross-sectional view of a stimulation device, in accordance with various disclosed aspects;

FIG. 2 illustrates a side view of the stimulation device of FIG. 1, in accordance with various disclosed aspects;

FIG. 3 illustrates a percutaneous electrode, in accordance with various disclosed aspects;

FIG. 4 illustrates another percutaneous electrode, in accordance with various disclosed aspects;

FIG. 5 illustrates an adaptor comprising a percutaneous electrode, in accordance with various disclosed aspects;

FIG. 6 illustrates an adaptor comprising two connectors, in accordance with various disclosed aspects;

FIG. 7A illustrates the stimulation device of FIG. 1 coupled with an adaptor, in accordance with various disclosed aspects;

FIG. 7A illustrates an exemplary electrical stimulation pulse in accordance with various disclosed aspects.

FIG. 8 illustrates a stimulation output pattern created by user intermittently contacting tissue. This output pattern is inconsistent in both the number of pulses per group and the time between successive groups of pulses.

FIG. 9 illustrates stimulation outputs of a stimulation device that generates consistent bursts or groups of pulses to innervated tissue, in accordance with various disclosed aspects;

FIG. 10 illustrates stimulation outputs of a stimulation device that generates bursts or groups of pulses at varying amplitudes to innervated tissue, in accordance with various disclosed aspects;

FIG. 11 illustrates stimulation outputs of a stimulation device that generates bursts or groups of pulses with a model of the evoked muscle contraction from a muscle consisting primarily of slow twitch fibers, in accordance with various disclosed aspects;

FIG. 12 illustrates stimulation outputs of a stimulation device that generates bursts or groups of pulses with a model of the evoked muscle contraction from a muscle consisting of primarily of fast twitch fibers, in accordance with various disclosed aspects;

FIG. 13 illustrates a stimulation device in accordance with various disclosed aspects utilize in a medical procedure as described herein;

FIG. 14 illustrates a system in which the stimulation device and detection device are decoupled, in accordance with various disclosed aspects; and

FIG. 15 illustrates a method of generating stimulation outputs via a stimulation device and detecting evoked muscle contraction, in accordance with various disclosed aspects.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present invention, examples of which are illustrated in the accompanying drawings. It is to be understood that other embodiments may be utilized, and structural and functional changes may be made without departing from the respective scope of the invention. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

It is noted that the various embodiments described herein may include other components and/or functionality. It is further noted that while various embodiments refer to a stimulator or stimulation device, various other systems may be utilized in view of embodiments described herein. For example, embodiments may be utilized in a variety of surgical procedures, other medical procedures or as a stand-alone procedure. As such, embodiments may refer to a particular surgical procedure for purposes of explanation. It is noted that aspects of embodiments, however, may be utilized for various other procedures or as a stand-alone procedure.

This disclosure generally relates to systems and methods that may improve nerve identification and assessment of neuromuscular function, such as during a surgical procedure or any other medical procedure. The terms “nerve” or “nerve tissue” generally refer to any portion of a nerve including, but not limited to, axons, axon terminals, somas, dendrites, fascicles or the like, unless context suggests otherwise. Moreover, aspects disclosed herein may be applicable to nerve tissue throughout a body, whether peripheral nervous tissue or otherwise. Further, while embodiments may reference a surgeon performing a particular action(s), it is noted that other clinicians or users, automated machines, or the like may perform such actions.

It is noted that described systems and methods may be utilized in combination with various systems and methods for safeguarding against nerve, muscle, and tendon injury during surgical procedures, other procedures or as a stand-alone procedure or confirming the identity and/or location of nerves, muscles, and tendons and evaluating their function and/or health or the function and/or health of muscles innervated by those nerves. The systems and methods are particularly well suited for assisting in monitoring or identification of nerves and muscles in order to assure nerve and muscle health and/or integrity during medical procedures using medical devices such as stimulation monitors, cutting, drilling, and screwing devices, pilot augers, and fixation devices, including without limitation, the system disclosed in U.S. Pat. No. 10,470,678 which is incorporated herein by reference. It is noted, however, that described systems and methods may utilize other devices that are not utilized during surgical procedures. Moreover, various disclosed aspects may be utilized independent of such systems and methods.

In an example, a surgeon, clinician or other medical professional may apply one or more leads to a user. The leads may be percutaneous, trans-cutaneous, surface electrodes, or leads directly attached to the nerve. The leads may be placed in a region suspected of nerve damage or diminished function, a region of possible nerve damage or diminished function, a region where an operation is to be performed or is being performed and/or a region of where a nerve is suspected to be. For instance, a procedure may be planned that requires operation at or near a nerve. This may put the nerve at risk for damage, temporary or permanent diminished function, or rehabilitation after the procedure.

By way of a non-limiting example in a transdermal delivery, an electrode may be implanted and may be wirelessly and operatively coupled with a surface coil or may be directly and operatively attached with the surface coil for power delivery and communication so that the electrode may apply the appropriate stimulation. The electrode may be positioned in any appropriate manner, such as through a needle, through surgical intervention or any other appropriate method.

In at least some embodiments, a surgeon or clinician may utilize a handheld stimulation device to generate an electrical stimulation for the purposes of locating, stimulating, and evaluating nerve or muscle, or both nerve and muscle integrity in numerous medical procedures. This may also include, but is not limited to, evaluating proximity to a targeted tissue region, evaluating proximity to a nerve or to identify nerve tissue, evaluating nerve health and/or integrity (i.e., following a traumatic or repetitive motion injury) to determine if a repair may be needed, evaluating muscle contraction to determine whether or not the muscle is innervated and/or whether the nerve is intact and/or whether the nerve is severed, and identifying specific nerve branches or fascicles for protection, repair or transfer prior to completing a surgical procedure. The stimulation device of the present disclosure may be particularly useful with fast twitch muscle where it can be difficult to identify, such as visually, a contraction and/or application of the electrical stimulation. By way of a non-limiting example, the stimulation device may be utilized in association with the following procedures: thyroid, parathyroid, brachial plexus surgery, facial procedures, peritoneum or the like. It should be understood, however, that these are merely examples of the procedures under which the stimulation device of the present disclosure may be used. The stimulation device may be utilized in any procedure for of locating, stimulating, and/or evaluating nerve or muscle, or both nerve and muscle integrity.

A surgeon may place an electrode or lead on or near the nerve to be stimulated and/or proximal to the site of injury, potential injury, or repair. The electrode may be percutaneous or non-percutaneous (e.g., surface electrode or implanted electrode). In some embodiments, the electrode may be percutaneously positioned while a component transmits energy to the percutaneously positioned electrode through tissue. It is noted that the transmitting component may be positioned on the skin at an appropriate location, i.e., it may be an external electrical stimulator (or pulse generator). In an aspect, a percutaneous lead may be taped or otherwise held in place on a patient's skin. This may allow for easy removal after prolonged stimulation. One exemplary embodiment of such comprises a patch that may be adhered to the skin of a patient. The patch may generally circumscribe the insertion point of the percutaneous lead and may allow a portion of the lead to extend therethrough. In this embodiment, a connector may be utilized to operatively couple the percutaneous lead with the stimulation device. Alternatively, the lead may be operatively coupled with the patch and the patch may include an adapter that operatively couples with the stimulation device. The patch may include an electrical path between the percutaneous lead and the adapter such that electrical stimulation may pass from the stimulation device through the patch and to the percutaneous lead.

In one example, electrical stimulation may be applied to determine a baseline level of nerve excitability before or at the start of a surgery. During or near completion of the surgery, a second test of nerve excitability may be conducted. This second test may be compared against the first. If the second test results in lower nerve excitability, the surgeon may be alerted to potential damage or impairment of a nerve or tissue. This series of tests may be done with a single electrode and stimulation device or may be accomplished with two or more electrodes or stimulation devices. Further still, the initial threshold test may be done with a different stimulation and the same lead or a different stimulation device and lead (such as a stimulation device with a permanently fixed lead) or with the same stimulation device and the same lead. The threshold test may comprise electrical stimulation to a nerve or nerves within a target tissue region to determine or measure the excitability of the nerve or nerves. In such situations, it may only be necessary to test nerve excitability to determine if the post procedure therapy is needed or additional procedures are needed. Threshold testing may be done at any time during the surgery, such as when a retractor is removed, a limb of a patient has a force applied to it, or the like.

As described herein, the parameters of the electrical stimulation for stimulation may be preprogrammed or may be set by a surgeon clinician. In at least one embodiment, the pulse duration or burst interval may be held constant (e.g., not adjusted) during the stimulation. In an aspect the electrical stimulation may be applied at generally between 2-100 Hz, 15-50 Hz, about 16-20 Hz, 2-5 Hz or 25-35 Hz. In some embodiments, the electrical stimulation applied may be as low as 0.1 Hz or as high as 1 kHz. In one exemplary embodiment that may be referred to, for example, as the primary faster stimulation, the electrical stimulation may be applied at between about 15-50 Hz. In another exemplary embodiment, the electrical stimulation may be applied at a slower burst frequency or burst rate/frequency where groups of the 16 Hz (or 32 Hz) and the overall stimulation may be applied at, for example, between 2-5 Hz. Stated another way, in some embodiments the frequency in the overall group of electrical stimulation and the frequency of the burst may be different, i.e., the burst frequency may be greater or less than the overall stimulation frequency. It should be understood that the reference to about means that the application of the stimulation may be in the ranges provided within a tolerance of 1 to 2 Hz. Further, reference to about a specified frequency means that defined frequency or ranges of frequency plus or minus between 0.1 Hz to 2 Hz.

In other embodiments, which may be combined with the embodiments above, the electrical stimulation for stimulation may be preprogrammed or may be set by a surgeon clinician such that the pulse duration is held constant and the amplitude may be adjusted. In these embodiments, the amplitude may be increased from 0.1 to 5 mA in any appropriate incremental increase. For example, the amplitude may be increased n mA where n equals any 0.1 mA increase between 0.1 to 0.5 mA. In sum, the electrical stimulation is made wider or made taller.

Stimulation systems described herein may deliver an electrical stimulation to peripheral nerves according to a defined stimulation pattern. An attachment (e.g., a percutaneous electrode lead attachment such as those described in U.S. Patent Application US2014/0073985A, which is incorporated by reference herein) may be coupled with a stimulation device. For instance, a surgeon may utilize a handheld stimulation device to apply stimulation. The surgeon may place one or more implantable leads at a position where the lead may stimulate nerves that may have damage, potential for damage, or may require evaluation or treatment after completion of the surgical operation.

As described herein, stimulation devices may be disposable or reusable. Described systems may allow a stimulation electrode to be placed intraoperatively, in close proximity to the nerve to be stimulated; allowing the lead to pass out of the tissue and attach, either permanently or selectively, to the stimulation device.

In at least one embodiment, an electrical stimulation system may comprise a stimulation device and an adaptor. The stimulation device may comprise a housing, a control circuitry operatively generating an electrical stimulation, wherein the control circuitry is disposed within the housing, and an operative element coupled with the housing and comprising at least one electrode. The housing may be configured as a hand-held housing that is suitable for use by a surgeon/clinician using a single hand. The adaptor may be selectively attached to the operative element, and may comprise a percutaneous lead electrically coupled to the stimulation device through the at least one electrode, the percutaneous lead insertable into a patient during a subcutaneous surgery and after the subcutaneous surgery and wherein the stimulation device is capable of applying electrical stimulation. The percutaneous lead may comprise strands of stainless-steel wire insulated with a biocompatible material.

In some traditional systems, a physician utilizes the stimulation device in a “touch and release” method where the physician applies and removes an electrode from innervated tissue. The physician monitors for informational feedback during tissue dissection or nerve exploration and repair. Typically, the stimulation device generates a constant or regular pulse, where pulses are generated with constant parameters (e.g., amplitude, pulse duration, etc.) and at regular rate or frequency (e.g., one pulse every t milliseconds, where t is a number). The “touch and release” method produces an irregular stimulation pattern, with both a variable number of pulses delivered each touch and a variable time between each touch.

It is noted that some systems may deliver stimulation at a frequency of 16 Hz (˜62 ms) or 32 Hz or other rates/frequency, e.g., between 0.1 Hz to 1 kHz. Depending on a patient and muscle fiber composition, the stimulation frequency may produce individual muscle twitches, an occasional “flutter” or partially fused contraction, or a tetanic contraction. The type of contraction depends on both the stimulation frequency (time between pulses), and the muscle fiber type. A tetanic contraction will be evoked in slow twitch muscle fibers at lower stimulation frequencies compared to fast twitch muscle fibers. This aspect of the underlying muscular physiology may be utilized by the stimulation pattern to improve detectability. For example, a stimulation frequency may generate a muscle twitch in a specific muscle that makes its detection easier than other traditional stimulation frequencies.

In described embodiments, stimulation devices may generate pulses according to stimulation patterns, where pulses may comprise non-uniform parameters (e.g., variable amplitudes, inter-pulse intervals, frequencies, etc.) or pulses are not consistently generated at a regular interval, e.g., the pulses may be non-regular. Disclosed embodiments generate pulses according to predefined patterns or non-regular patterns to create detectable twitches or evoked potentials that may be distinguishable from or detectable in the presence of inadvertent muscle twitches, flutters, tetanic contraction, or in situations where tissue innervated by the evaluated nerves are small, weak, or difficult to palpate. In examples, false positives, false negatives, difficulty in readings, or other errors may be reduced.

According to at least some examples, described systems and methods may utilize evoked potential sensors to automate detection of electrical stimulation by computational algorithms stored on a computer readable medium and executed by a processor. Such algorithms may guard against the false alarms experienced by some traditional devices and may reduce dependence on absolute threshold such that detectability of stimulation may be improved. For instance, a stimulation device may comprise a handheld-intraoperative nerve stimulator used for nerve location and identification with programmed stimulation patterns. The stimulation pattern may provide improved detectability by humans and machine algorithms over continuous or manually controlled stimulation. It is noted that that stimulation may be applied by a physician as needed, applied for automated detection for continuous intraoperative nerve monitoring, or the like.

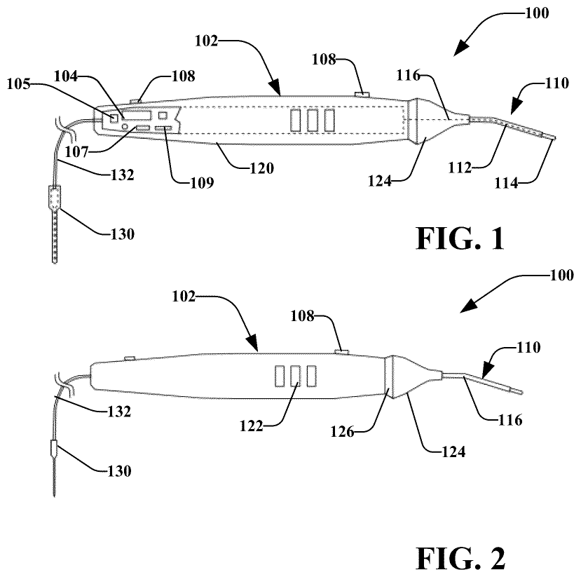

Turning now to FIGS. 1 and 2, there is a stimulation system 100 that may comprise a stimulation device 102 configured for locating, monitoring, and stimulating tissue and other structures throughout the body. The stimulation system 100 may be utilized for locating and identifying tissue and safeguarding against tissue and/or bone injury during surgical procedures. In another aspect, the stimulation device 102 may generate and apply stimulation pulses according to a pattern that is not a continuous simulation (e.g., regular pulses spaced temporally equidistance from each other or non-regular pulses). The stimulation system 100 may identify the level of stimulation required to identify (whether manually or through sensors) before a desired response is evoked (e.g., a tetanic stimulation for slow twitch muscle fibers, or an occasional “flutter” for fast twitch muscle fibers to cause movement of a limb such that the entire limb doesn't move). The amount energy required to reach the desired response may then be recorded and this information utilized to evaluate the nerve being stimulated or the muscle innervated by the nerve being stimulated.

The stimulation device 102 may include or be coupled with one or more attachments or operative elements including, for example, a probe 110 (e.g., which may be blunt, needle-like, etc.), a cutting device, a drilling or screwing device, a pilot auger, and a fixation device. It is noted that attachments may be removable, attachable, or permanently affixed to the stimulation device 102. It is noted that while embodiments may describe use of a particular attachment (e.g., probe 110) for simplicity of explanation, the various embodiments may utilize other types of attachments.

In an exemplary embodiment, stimulation device 102 comprises control circuitry 104, disposed in a housing 120, that may apply an electrical stimulation to a desired tissue region. The control circuitry 104 may be coupled to a power source, such as a battery, power mains, or the like. The control circuitry 104 may generate the electrical stimulation with desired parameters, as described herein. In an aspect, a user may adjust parameters and/or control the control circuitry 104 to generate the electrical stimulation via one or more user interfaces 108, which may comprise at least one of a switch, button, slide, touch screen, or the like, such as a momentary button switch. In an example, the user may interact with the momentary button switch to alternate between stimulation modes, such as “constant stimulation,” “burst stimulation,” or “wave” mode. It is noted that a single push may switch to a different mode so that a user may cycle between modes by pushing the button. In other examples, applying stimulation without pushing the momentary button switch may activate a first mode and applying the stimulation with pushing the momentary button switch may activate a second mode.

For instance, a user may grasp the stimulation device 102 via the housing 120. The housing 120 may comprise a handheld device that is graspable by a single hand of a user. Further, the housing 120 may be of such a configuration that it can be completely within the surgical field. This may make it easier to use during a surgical procedure. The stimulation system 100, however, may also be used outside of the surgical field or completely outside of the confines of a surgery. The housing 120 may include gripping portion 122. The gripping portion 122 may comprise indents, protrusions, elastomeric material, roughened material or other features that may aid in the user grasping the stimulation device. The gripping portion 122 of the housing 120 may include an over molded portion that may comprise all or part of the length of the housing 120. In an aspect, the over molded portion may comprise a thermoplastic elastomer material. It is noted that gripping portion 122 may be removable, attached to, or integrally formed with the housing 120.

In an example, a user may position the probe 110 so that an uninsulated or stimulating portion or electrode 114 is at a desired location. This may include positioning the electrode 114 relative to a nerve, a tissue or attachment. The user may interact with one or more of the user interfaces 108 to control delivery of electrical stimulation, generated by the control circuitry 104, to the desired tissue region. The gripping portion 122 may aid in a user's efforts to hold the stimulation device 102. In an aspect, the control circuitry 104 communicates the electrical stimulation to the stimulation probe 110 via a lead 112 that may travel through an insulated portion 112 of the probe to the uninsulated portion 114.

It is noted that the probe 110 may comprise one or more flexible materials (e.g., metal, plastic, etc.) so that a user may bend or otherwise manipulate the probe 110. In another aspect, the stimulation device 102 may comprise a nose cone 124 that may be flexible or rigid. An operative element (e.g., probe 110) may extend from the proximal end of the nose cone 124. The user may apply pressure to the nose cone 124 so that it moves or otherwise manipulates the probe 110. This may allow a surgeon or other user to position the uninsulated portion 114 at a desired position of a targeted tissue region. For example, the uninsulated portion 114 of the probe 110 is positioned in electrically conductive contact with at least one of muscle, nerve, or other tissue.

A flexible nose cone 124 may allow the surgeon to use either a finger or a thumb positioned on the nose cone 124 to make fine adjustments to the position of probe 110 at the targeted tissue region. The surgeon may grasp the housing 120 with the fingers and palm of the hand, and position the thumb on the nose cone 124, and with pressure applied with the thumb, cause the probe 110 to move while maintaining a steady position of the housing 120. This flexible nose cone 110 may allow for increased control of the position of the probe 110 with the movement of the surgeon's thumb (or finger, depending on how the stimulating probe is held). In another aspect, the nose cone 124 may comprise gripping components, such as ribs, indents, roughened surfaces, or the like.

It is noted that the nose cone 124 may comprise a single piece or it may comprise one or more pieces attached together. For example, nose cone 124 may comprise an inner portion that may include thermoplastic material having flexibility (e.g., LUSTRAN.® ABS 348, or similar material), and an outer portion that may comprise a softer over molded portion and may be made of a thermoplastic elastomer material having flexibility (e.g., VERSAFLEX.™. OM 3060-1 from GLS Corp). It is noted, however, that nose cone 124 may be generally rigid in at least some embodiments.

While described as a “cone” nose cone 124 may comprise a generally tapered shape or different shapes (e.g., rounded, squared, prism, conical, etc.). Moreover, in embodiments, stimulation device 102 may not comprise a nose cone 124, such that probe 110 extends directly from housing 120.

As described herein, an electrical stimulation may flow from the stimulation device 102 through the lead 112 to the probe 110, which may act as an electrode. The electrical stimulation may comprise a desired pattern of pulses, comprising specified frequencies, duration, amplitudes, or the like.

In those embodiments in which the stimulation system 100 is utilized for threshold testing, the stimulation system 100 may include an actuator that is operatively coupled with the control circuit 104. The actuator may automatically initiate the threshold testing. The actuator may comprise any appropriate mechanism, such as a button, rotary wheel, a touch button (screen portion) having a proximity sensor for determining if the touch button is being contacted, a slide or any appropriate configuration. For example, in use when the probe 110 is in the operative position, the actuator may be actuated to begin the threshold testing. The stimulation may start at 0.5 mA at 10 μs and continue to 2.0 mA and 200 μs. In one example, the stimulation may have a pulse duration of 10 μs, and every period of time, the pulse duration may increase by 10 μs until an applicable response is initiated, e.g., a tetanic response for slow twitch muscle fibers, or a “flutter” for fast twitch muscle fibers. Once the appropriate response occurs or is detected (whether visually by a clinician or by an applicable sensor), the threshold test is stopped and the details of the stimulation that invoked the response is recorded, whether by a separate device or by the stimulation system 100. This test may be repeated again to determine the stimulation that invokes the applicable responses in the second instances. This may be compared to the first threshold test to determine the functionality and/or health of the nerve and/or muscle being innervated.

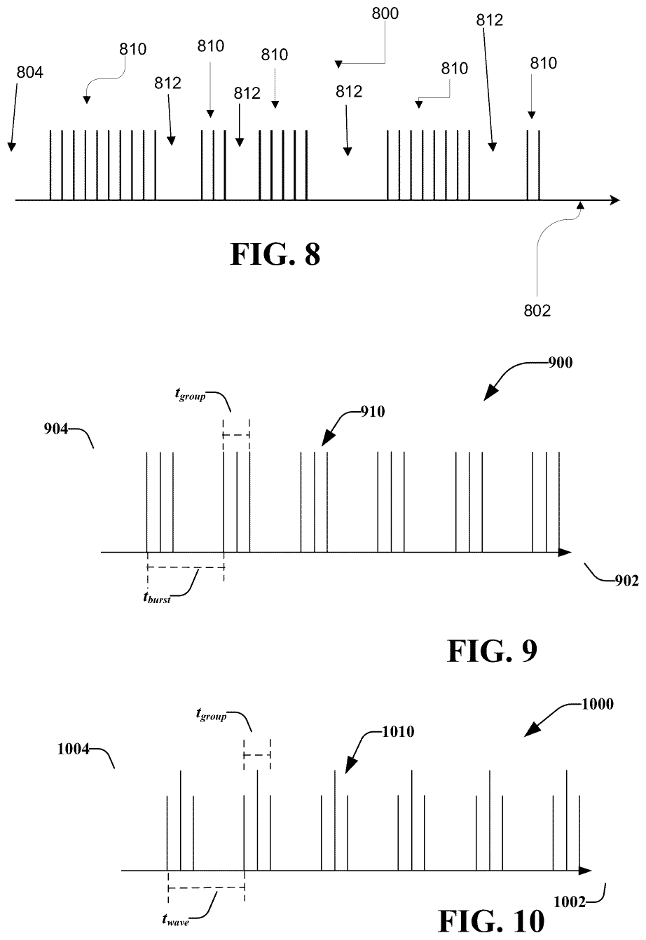

Referring to FIG. 8, there is a graph 800 illustrating pulses 810 generated at irregular intervals due to “touch and remove” method as shown by the x-axis 802, and at constant amplitude as shown by the y-axis 804. This output of stimulation device 102 may generally be utilized in “touch and remove” mode, where physicians apply the probe 110 or the lead 112 at a desired location at desired times. When, the physician touches tissue with the probe 110, stimulation is generated in pulses 810 until the physician removes the probe 110 at period 812. If the physician had not removed the probe or turned off stimulation during the period 812, stimulation would have continued to generate pulses at the same rate/frequency during period 812. The physician removes and applies the stimulation during operation or evaluation. It is noted that this constant amplitude or regular intervals of pulses 810 may result in difficulties in reading or detecting stimulation under certain circumstances. For instance, stimulation at a rate/frequency of 16 Hz (˜62 ms) or other rates/frequencies (e.g. 8, 24, or 32 Hz) may result in stimulation frequency that results in tetanic stimulation for slow twitch muscle fibers, or a occasionally a “flutter” for fast twitch muscle fibers.

FIG. 9 illustrates a graph 900 of output pulses 910 generated by the stimulation device 102 in a stimulation pattern referred to herein as a “burst mode.” As shown, the x-axis 902 illustrates time and the y-axis 904 illustrates the amplitude. It is noted that the stimulation outputs are illustrated graphically at a nominal 16 Hz (62 ms between pulses), but other rates/frequencies may be utilized, including, without limitation 32 Hz, 5 Hz-64 Hz. In this example, the control circuitry 104 of stimulation device 102 includes memory comprising instructions that, when executed by a processors, cause the stimulation device 102 to generate pulses in groups with time t_{group} between the first and last pulse of each group and a larger time t_{burst} between the first pulse of a first group and the first pulse of a second group.

The control circuitry 104 may generate pulses in groups of i pulses, where i is a number (e.g., 3, 5, 7, etc.) and at a particular frequency and amplitude. Such stimulation patterns may induce repeated slow-twitch contractions that may be more readily detectable and distinguishable from a constant pulse rate/frequency, from inadvertent muscle twitches, or the like. The time between the pulse groups (i.e., the time from the first pulse of a burst to the first pulse of the next burst) by the time period t_{burst} may allow for recovery of slow twitch muscles between pulse groups. According to at least one example, an effective twitch rate/frequency of 1.6 Hz (groups of 5 on 5 off), 2 Hz (groups of 4 on 4 off) and/or 2.7 Hz (groups of 3 on 3 off) when stimulus frequency is 16 Hz may be monitored or detected. This may allow for improved detection and monitoring or nerve health or function. According another example, an effective twitch rate/frequency of 3.2 Hz (groups of 5 on 5 off), 4 Hz (groups of 4 on 4 off) and/or 5.3 Hz (groups of 3 on 3 off) when stimulus frequency is 32 Hz may be monitored or detected. This may allow for improved detection and monitoring or nerve health or function. These are merely exemplary. Any frequency between 0.1 Hz to 1 kHz and/or between 5 Hz to 64 Hz may be utilized. Further, while the pattern is described as a number of pulses on and a number off, the pulses on may be x on pulses where x is any number (e.g., 1-100) and the number of pulses off may be y pulses where y is any number (e.g., 1-100) and where x and y may be the same number or a different number.

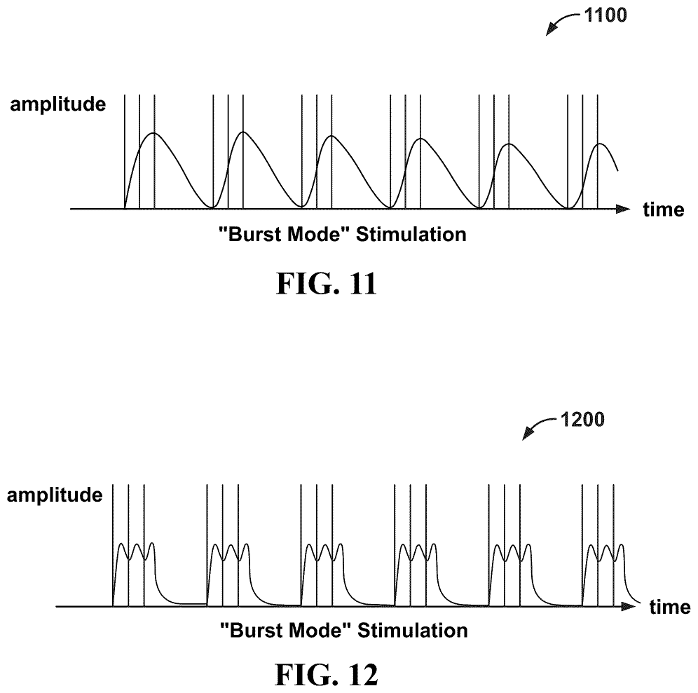

FIGS. 11-12, further illustrate physiologic muscle response from a muscle primarily composed of slow twitch fibers, FIG. 11, versus a muscle composed mostly of fast twitch fibers, FIG. 12. The burst pattern may produce different types of muscle contractions depending on the muscle fiber type and composition. The burst pattern may be optimized to produce specific types of evoked contractions to aid in detectability, e.g., a stronger tap to produce an easier to perceive contraction.

FIG. 10 illustrates a graph 1000 of output pulses 1010 generated by the stimulation device 102 in a stimulation pattern referred to herein as a “wave mode.” As shown, the x-axis 1002 illustrates time and the y-axis 1004 illustrates the amplitude. Similar to the burst mode, control circuitry 104 of stimulation device 102 includes memory comprising instructions that, when executed by a processors, cause the stimulation device 102 to generate pulses in groups with time t_{group} between the first and last pulse of the group and a larger time t_{wave} between the first pulse of a first group and the first pulse of a second group. In the wave mode, the parameters for different pulses may be adjusted, such that the parameters of pulses within one group or between different groups vary. FIG. 10 illustrates changes in amplitude between groups of three pulses where a first pulse comprises a first amplitude, a second pulse comprises a second amplitude greater than the first amplitude (although it could be less in other embodiments), and a third pulse comprises a third amplitude lesser (although it could be greater in other embodiments) than the second amplitude or generally equal to the first amplitude. The foregoing are merely exemplary. Other variations may be utilized such that stimulation parameter (e.g., pulse duration, base frequency, and pulse train duty cycle) may be varied to provide different stimulation patterns. For example, stimulating at a higher frequency but still in the realm of physiologically appropriate (e.g. 32 Hz modulated at 5.3 Hz with a 50% on-off duty cycle) would allow detection of dual frequency components in half the time required for 16 Hz or 32 Hz, as applicable.

Turning back to FIG. 1, the stimulation device 100 may control or allow a user to select a particular stimulation pattern or mode. For instance, control circuitry 104 may receive input from a user via the user interface 108 as described herein. The control circuitry 104 may select a specified mode or stimulation pattern and identify the mode to a user. This may allow improved detection of stimulation through palpation, and selection of different modes to address particular physiological differences between patients.

The stimulation system 100 may include one or more other electrodes, such as a return electrode, as described herein. For instance, in monopolar operation, a return electrode (or indifferent electrode) provides a return path for electrical stimulation passing through the tissue, and returning to the stimulation device 102. It is noted that stimulation system 100 may operate in a monopolar, bipolar or other configurations, as described here as well as elsewhere in this disclosure.

In various embodiments, the control circuitry 104 may generate the electrical stimulation to operatively generate a physical motor response of a tissue (e.g., muscle, innervated muscle, nerve, etc.). The physical motor response may indicate whether the electrical stimulation was delivered and/or whether a sufficient electrical stimulation was delivered. For example, the motor response may include a physical motor response (e.g., twitching or contraction).

In at least one embodiment, embodiments the system 100 may detect stimulation through a detection device. Any appropriate embodiment of detection device may be utilized without departing from the present teachings, including, without limitation return electrode, EMG sensor, a cuff on an endotracheal tube that detects pressure changes, visual movement via a camera or AI system, or other device as described herein).

In another aspect, the stimulation device 102 may generate one or more visual or audio signals (e.g., via a speaker (not shown)), which indicate to the surgeon the status or diagnostic information. For instance, stimulation device 102 may comprise an indicator light 126. The indicator light may comprise one or more light sources, such as a light emitting diode (LED). In an aspect, the indicator light 126 may comprise a translucent (e.g., semi-translucent, fully translucent, etc.) surface that operatively shines or disperses light from an internal light source (not shown). In an aspect, the light source may generate light in one or more colors (e.g., green, yellow, blue, red, etc.), patterns (e.g., blink rate, pattern of colors, etc.), or the like. According to embodiments, the status or diagnostic information may indicate whether the electrical stimulation was delivered, whether a sufficient electrical stimulation was delivered (i.e., the requested amount of or the designated electrical stimulation was delivered), a selected stimulation pattern (e.g., may light or blink when pulses are sent), or the like. For example, the status or diagnostic information may indicate that an electric stimulation was returned from tissue, which may indicate sufficient proximity, contact, and/or delivery of the electrical stimulation requested via an operative element (e.g., probe 110). In another aspect, the indicator light 126 may indicate that the stimulation device 102 is on/off, producing or not producing an electrical stimulation, a selected pattern of stimulation, or the like. Still further, the indicator light 126 may indicate that the electrical stimulation requested from the stimulation device 102 by the user completes the electrical flow path going through the operative element (e.g., probe 110) and the targeted tissue region and back through the return electrode at a specified stimulation level equivalent to a control setting selected by the user on the stimulation device 102. The indicator light may provide, in response to determining whether the electrical stimulation completes the electrical flow path going through the operative element (e.g., probe 110) and the targeted tissue region and back through the return electrode in accordance with the specified stimulation level, a first indication signal for confirming delivery of the electrical stimulation to the targeted tissue region through the operative element and back through the return electrode completing the electrical flow path at the specified stimulation level. The indicator light 126 may also provide, in response to determining whether the electrical stimulation going to the operative element and the targeted tissue region and back through the return electrode is not at the specified stimulation level, a second indication signal to the at least one indicator for indicating a failure of delivery of the electrical stimulation to the targeted tissue region at the specified stimulation level through the operative element to the targeted tissue region and back through the return electrode. The indicator light 126 incorporated with the housing may provide reliable feedback to the surgeon/user as to the request and delivery of stimulus current. Moreover, in examples, the indicator light 126 may indicate whether a returning electrode receives response signals that match the pattern of stimulation.

In an example, the indicator light 126 allows the surgeon to confirm delivery of stimulus current, or more specifically, the pre-selected or desired stimulus current, and pre-selected stimulation pattern (e.g., pulse pattern) to tissue. Through the use of different tones, colors, different flash rates, etc., the indicator 126 allows the surgeon to confirm that the uninsulated tip 114 is in place, the stimulation device 102/instrument is turned ON, and that stimulus current is flowing with sufficient (e.g., the desired) delivery to tissue, which may be determined by identifying induced responses related to the stimulation pattern. Thus, the surgeon has a much greater confidence that the desired stimulation amplitude is actually being delivered to the nerve, as in the case of a nerve transfer of nerve graft, a muscle contraction will not be observed since the nerve is no longer in continuity. These indicators can be checked periodically to ensure stimulation (e.g., the desired stimulation) is being delivered for the desired duration (e.g. between about 1 minute and one hour). In these embodiments, the indicator 126 is determining more than just whether the operative element is providing stimulation, it indicates whether or not the electrical stimulation completes the electrical flow path going through the operative element (e.g., probe 110) and the targeted tissue region and back through the return electrode at a specified stimulation level based on a control setting selected by the user on the stimulation device 102, as well as reducing false positives or other errors due to inadvertent muscle twitches, tetanic contraction, in situations where tissue innervated by the evaluated nerves are small, weak, or difficult to palpate.

As another example, in use the indicator 126 may be configured to illuminate continuously in one color when the stimulation device 102 is turned on but not in contact with tissue. After contact with tissue is made, the indicator 126 may flash (i.e., blink) to indicate that stimulation is being delivered (as described above). If the stimulation has been requested, i.e., the stimulation probe has been turned on, but there is no stimulation being delivered because of a lack of continuity between the probe 110 and the return electrode 130, or an inadequate connection of the probe 110 or the return electrode 130 to the patient tissue, or the stimulation device not delivering to the tissue the desired/pre-selected electrical stimulation, the indicator 126 may illuminate in a different color, and may illuminate continuously or may flash.

As described herein, the indicator 126 may comprise a ring that provides a visual indication around at least a portion, and desirably all of the circumference of the stimulation device 102 generally near the nose cone 124. A ring indicator may be an element of the gripping portion 122, or it may be an element of the flexible nose cone 124, or the ring indicator may be positioned between the gripping portion 122 and the nose cone 124. The ring may also include a reflective element to improve and focus the illumination effect of the light emitting source, e.g., one or more LEDs. The ring and the reflective element may be a single component, or more than one component. Audio feedback also makes possible the feature of assisting the surgeon with monitoring nerve integrity during surgery.

While stimulation device 102 is described as generating an indication, it is noted that various other components of the stimulation system 100 may generate all or part of the indication. For instance, the stimulation device 102 (or a separate device) may monitor delivery of the electrical stimulation. The stimulation device 102 may transmit status and diagnostic information (e.g. delivered current, stimulation duration, contraction presence, or the like) to a separate device (e.g., laptop, wearable electronic device, cellular phone, tablet, computer, speakers, light source, or the like). In an aspect, the stimulation device 102 may include a communication component that may be wired or wireless. For example, the stimulation device 102 may include a wireless transmitter/receiver configured to communicate via one or more communication protocols (e.g., Wi-Fi, BLUETOOTH, NFC, etc.).

In embodiments, stimulation device 102 may comprise a hand-held stimulation device. Housing 120 may be generally tubular, hexagonal, or other elongated shape. According to an aspect, the housing 120 may be ergonomic and sterile for use in operative procedures. For instance, the stimulation device 120 may be packaged in a sealed container that may allow a surgeon to open and use the stimulation device 120 without the need for sterilization. It is noted, however, that parts of the stimulation system 100 may be sterilized, such as probe 110. In another aspect, the stimulation device 120 may comprise a single use instrument for use during surgical procedures to identify nerves and muscles, muscle attachments, contract muscles to assess the quality of surgical interventions or the need for surgical interventions, evaluate the function and/or health of nerves already identified through visual means, or provide prolonged stimulation of a nerve.

The stimulation device 120 may be sized small enough to be held and used by one hand during surgical procedures, and may be ergonomically designed for use in either the left or right hand. In an embodiment, the stimulation device 120 may have a width of about 20 millimeters to about 30 millimeters, and desirably about 25 millimeters. The length of the stimulation device 120 (not including an operative element) may be about 18 centimeters to about 22 centimeters, and desirably about 20 centimeters. An operative element (e.g., probe 110) may also include an angle or bend 116 to facilitate access to deep as well as superficial structures without the need for a large incision. As illustrated, the bend 116 may be generally downward, relative the directions shown in FIGS. 1 and 2. In an aspect, this may allow a surgeon to maintain a line of sight with target tissue and/or the uninsulated portion 114.

In one or more embodiments, as described here as well as elsewhere in this disclosure, an operative element may be monopolar or bipolar. For instance, probe 110 may be monopolar. A return electrode 130 may be coupled to control circuit 104 via an insulated wire 132. The return electrode 130 may comprise any of a variety of electrode types (e.g., paddle, needle, wire, or surface electrode). In another aspect, the stimulation device 102 may be bipolar and may comprise a return electrode in the probe 110 or other operative element.

User interfaces 108 may allow a user to turn ON/OFF the stimulation device 102 (or set to standby), and may allow a user to control the electrical stimulation amplitude selection within a predefined range (e.g., 0.1 0.5, 1.0, 2.0, and/or 20 mA). In configurations, user interface 108 may be a four or five position switch. It is noted that the user interface 108 may allow for selection and change of frequencies within a range. Before the first use of the stimulation device 102, the user interface 108 is in the OFF position and keeps the stimulation probe off. After the user interface 108 has been turned ON (e.g., by moving the switch 155 to an amplitude selection), the OFF position now corresponds to a standby condition, where no stimulation would be delivered. In one embodiment, once the stimulation device has been turned on, it cannot be turned off, it can only be returned to the standby condition and will remain operational for a predetermined time, e.g., at least about seven hours. This may allow the stimulation device 102 to be only a single use device, so it cannot be turned OFF and then used again at a later date. It is noted, however, that some embodiments may allow the user to turn off the stimulation device 102 after it has been turned on. In one example, the user interface 108 may allow for selection of “prolonged stimulation.” Once prolonged stimulation has been selected, the stimulation device 102 may disable user control of certain stimulation parameters, may allow the stimulation device 102 to be turned off, or may turn off after a certain time in the prolonged stimulation mode (e.g. 1 hour).

Further still, the stimulation device 102 may shut off automatically after a pre-defined period of time, such as by way of a non-limiting example, two hours or more, one hour, thirty minutes, fifteen minutes, ten minutes, five minutes, one minute or thirty seconds. Any pre-defined time may be utilized. In these embodiments, the stimulation device 102 may include a timer 109. The timer 109 may be operatively coupled with or be integral to the control circuit 104. The time 109 may be triggered upon the stimulation device 102 being turned on by the user as previously described. For example, the user may actuate, touch or otherwise engage the user interface 108 to turn the stimulation device 102 on to an active state. The time 109 may then count down a predetermined amount of time and at the conclusion of the predetermined amount of time, the stimulation device 102 may turn off, such as without human intervention.

In some embodiments, the time 109 may continue to run for a second predetermined amount of time, which may be greater than, less than or equal to the predetermined amount of time. At the conclusion of the second predetermined amount of time, the stimulation device 102 may automatically turn back on to apply electrical stimulation or it may utilize the indicator 126 to identify to the user/surgeon that the second predetermined time has expired and the stimulation device 102 may be turned on to provide stimulation; during the second predetermined amount of time, the stimulation device 102 may not be capable of turning on. In this case, the user/surgeon or even the patient may utilize the user interface 108 to manually turn the stimulation device 102 on to apply electrical stimulation. This may allow the stimulation device 102 to provide an automatic dosing regimen.

In some embodiments, once the predetermined amount of time has elapsed, the stimulation device 102 may be unable to be turned back on to apply stimulation. This essentially results in the stimulation device becoming a single use device.

Still further, the stimulation device 102 may include a pulse counter 105 of any appropriate configuration. The pulse counter 105 may count the number of pulses applied by the stimulation device 102 and turn off after a predetermined number of pulses has been applied. The pulse counter 105 may be operatively coupled with or integral to the control circuit 104. Upon occurrence of the predetermined number of pulses, the stimulation device 102 may automatically turn off. The stimulation device may then use a time 109 as described above to turn on after a predetermined amount of time to apply electrical stimulation or it may utilize the indicator 126 to identify to the user/surgeon that the predetermined time has expired and the stimulation device 102 may be turned on to provide stimulation. In this case, the user/surgeon or even the patient may utilize the user interface 108 to manually turn the stimulation device 102 on to apply electrical stimulation. This may allow the stimulation device 102 to provide an automatic dosing regimen. In fact, this process may be repeated for any number of stimulation cycles whereby the stimulation device 102 is automatically (without human intervention) on for the predetermined number of pulses and off for a resting amount of time. Each cycle may have a different predetermined number of pulses, such as a cascading amount of pulses, or may have the same predetermined number of pulses. The resting amount of time may be constant or may vary depending upon the dosing schedule, e.g., it may increase or decrease each successive period. The stimulation device 102 can apply a dosing regimen utilizing cycles of the predetermined number of pulses that have different stimulation patterns (or in some embodiments, the same stimulation pattern). The predetermined number of pulses and the resting predetermined amount of time may comprise any appropriate period, such as for example, one or more hours, thirty minutes, twenty minutes, fifteen minutes, ten minutes, one minute or thirty seconds. The present teachings are not limited to a specific time and/or number of pulses.

In some embodiments, once the predetermined number of pulses has occurred, the stimulation device 102 is unable to be turned back on to apply stimulation. This essentially results in the stimulation device being a single use device.

In some embodiments, the pulse counter 105 may include a sensor or plurality of sensors that can determine the number of pulses generated from the stimulation device 102 (or more specifically from the probe 110). Still further, the pulse counter 105 may include a stimulation sensor or a plurality of stimulation sensors that sense or determine the number of pulses actually delivered through the probe 110 from the stimulation device 102 to the targeted tissues (or nerve(s)) of the patient. This stimulation sensor is able to determine if an electrical pulse emitted from the probe 110 is actually delivered to the patient. In this embodiment, therefore, the pulse counter 105 will count the total number of pulses actually delivered to the patient. If a pulse, for any reason, is not delivered to the patient, the pulse counter 105 will not count it and such pulse will not count toward the predetermined number of pulses the pulse counter 105 is counting.

Further, the system may be able to compare the number of electrical pulses sent from the device against the number of pulses actually delivered to the patient. This may be utilized to determine the function of the device and/or the stimulation parameters being sent. Such sensors may comprise an electrode attached to the nerve to determine if there had been a pulse delivered to the nerve. The electrode may be configured to pick up a generally weak signal amid “background noise” in the patient. Still further, the sensors may be configured to identify if a certain patient stimulation response is recognized during the stimulation. For example, the sensor may be able to determine a tetanic response for slow twitch muscle fibers, or a “flutter” for fast twitch muscle fibers. If the expected response occurs, the stimulation system 100 may use any of the aforementioned indicators to indicate that the expected response occurs. Similarly, the stimulation system 100 may utilize any of the indicators to indicate whether the expected response did not occur. This information may be utilized to determine the health and/or functionality of the nerve being stimulated.

Still further, the pulse counter 105 may include an evoked potential sensor 107 or plurality of evoked potential sensors that can determine the number of evoked responses generated from the stimulation device 102 (or more specifically from the probe 110) through the nerve or nerves of the patient. The evoked potential sensor 107 or sensors may comprise an accelerometer, an electrode (of any appropriate configuration) or the like. In an example, the system may include one or more leads for stimulation and an evoked response sensor(s) 107 comprising one or more leads for recording activity, such recording electrical activity. The evoked response sensor 107 may determine generation of an evoked potential from the nerve or nerves being stimulated through the probe 110 by the stimulation device 102, or evoked activity in innervated muscle. The evoked response sensor 107 is able to determine if an electrical pulse emitted from the probe 110 is actually delivered to the patient and that it generates an evoked response potential. For example, the evoked response sensor 107 may be placed on, coupled to or otherwise attached to a location on a patient whereby the evoked potential can be detected upon application of the stimulation. In such a case, an electrode may be located in an area proximal and/or distal to the location of the stimulation in the patient where the nerve being stimulated innervates. In an example, evoked potentials going to the spinal cord, going to muscle tissues, or going in other directions may be recorded. The electrode may, alone or in combination with other components (e.g., a processor and memory), determine if an evoked potential is generated and sends a signal to the pulse counter 105 indicating that the evoked potential was generated such that the pulse counter 105 can count the generation of the evoked potential. Still further, the evoked potential sensor 107 may comprise an accelerometer that may be placed on, coupled to or otherwise attached to the patient to determine if an evoked potential was generated through the stimulation from the stimulation device 102. The accelerometer may be useful if the stimulation being applied is strong enough to invoke a muscle contraction that the accelerometer can sense and send a signal to the pulse counter 105 indicating that an evoked potential was generated. Regardless of the configuration of the evoked potential sensor 107, the pulse counter 105 will count the total number of evoked potentials generated in the patient. In this condition an indicator may inform the user that adequate stimulation is not being delivered and therapy paused until stimulation parameters are adjusted, or stimulation adjustment may be automatically done by the software until evoked potentials are observed. In some embodiments, the indicator may be triggered after a given number of not generated evoked potentials are not identified within a given time frame or otherwise missed (e.g., missed x consecutive, missed i out of the last j, missed x within the past y milliseconds, etc.).

It is noted that in some embodiments, one or more of the pulse counter 105, evoked potential sensor 107, or timer 109 may be comprised in a single device, in multiple device, within dedicated hardware and software, or as software stored within a memory and executed by a processor.

As described herein, evoked potential sensor 107 may be part of or coupled to the stimulation device 102, a separate component from stimulation device 102, or communicatively coupled to the stimulation device 102. Evoked potential sensor 107 may include an electromyography (EMG) device (e.g., endotracheal tube for intraoperative nerve monitoring of the recurrent laryngeal or vagus nerves), accelerometers, pressure sensors for detecting stimulation of innervated tissue (e.g., detecting pressure of vocal folds for stimulation of recurrent laryngeal nerve, pressure-sensitive conductive sheets such as VELOSTAT/LINQSTAT), air chambers (e.g., “balloon” cuff) and pressure sensors,) used (for example) around the perimeter of an endotracheal tube, subcutaneous probes inserted into the surgical wound for detection of EMG or vibration, or other devices. In an example, evoked response sensor 107 may allow for sampling and recording stimulation for “real-time” processing.

In another aspect, evoked potential sensor 107 may be communicatively coupled with the control circuitry 104 of the stimulation device 102, or to a processor of another device that may be a stand-alone device. It is noted that examples refer to stimulation device 102 as communicatively coupled to the evoked potential sensor 107 at least for sake of brevity. By utilizing disclosed stimulation patterns, the stimulation device 102 detect stimulation and evoked potential while reducing or eliminating false or potentially false readings. For instance, stimulation device 102 may be programmed to identify or detect readings from evoked potential sensor 107 to correspond with a selected stimulation pattern (e.g., burst or wave stimulation patterns). In an aspect, utilizing frequency-based detection rather than using absolute threshold to establish whether the nerve is stimulated may allow detection of less direct stimulation, stimulation at lower currents, and may reduce false alarms (e.g. from respiration or surgical manipulation).

In another example, stimulation patterns may include a plurality of frequencies to induce evoked potential. For instance, the stimulation device 102 may utilize two frequencies (e.g., in the burst mode example above/below, 16 Hz and 2.7 Hz (or 32 Hz and 2.7 Hz)), such that a muscle twitch output may reflect both frequency components. This allows the stimulation device 102 to detect whether stimulation has caused a muscle twitch. Techniques may include a using a Fourier transform or a Goertzel filter to identify whether measured responses pertain to a stimulation pattern.

In another example, the system 100 may allow for continuous monitoring of evoked potential without requiring user identification or observation or muscle twitch. For instance, system 100 may allow for machine based or automated detection of stimulation during operations, such as intraoperative nerve monitoring). Nerves may be verified as intact by stimulation device 102 periodically sending stimulation and examining the response, such as measured through evoked potential sensor 107. Described embodiments provide for more detectable stimulation patterns that can be monitored in an automated fashion with less or no technician involvement. As such, system 100 may allow for stimulation device 102 or a combination of devices to act as stimulator and a detector, to be placed intraoperatively in an uncoupled fashion. It is noted that such devices may be independent, disposable devices, reusable devices, limited use devices, or the like.

In at least one example, the stimulating device 102 may deliver a burst stimulation pattern, such as a group of pulses every 5 seconds, with the detecting device (whether contained within or separate from the stimulation device 102) may indicate a single tone that stimulation was read. After a continued connection is established (say 5 successful stimulation pulse groups), the device may reduce its frequency of indicating success while still monitoring at the faster rate/frequency. If the specific pattern (16 Hz bursts at 1.6 or 2.7 Hz or 32 Hz bursts at 1.6 or 2.7 Hz) is not detected every (e.g.) 5+/−2 seconds, the device may indicate a second tone pattern (e.g. high-low). In an example, the detecting device may include a watch-dog timer circuit or software executing a watch-dog timer program.

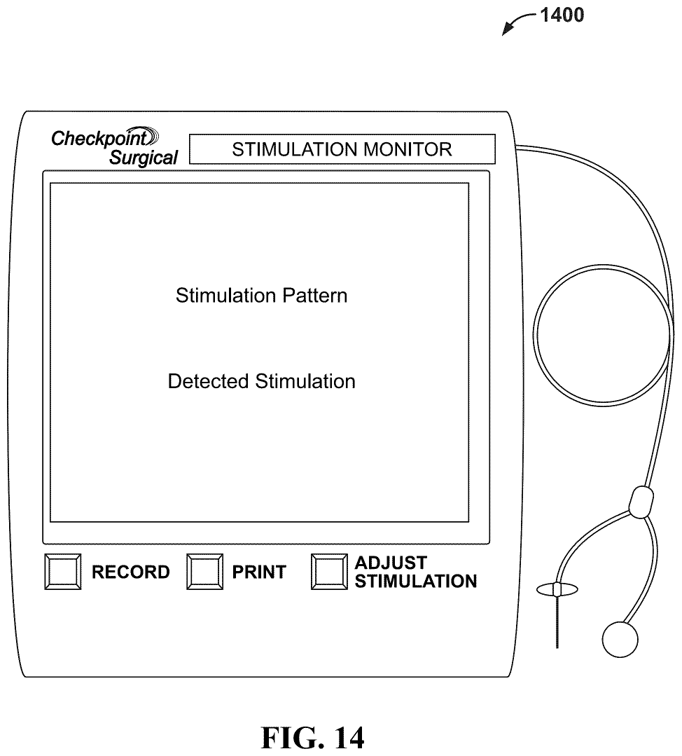

In some examples, the stimulating device 102 or another device may comprise an interface for indicating during continuous monitoring of nerve function—this may be used to determine nerve health. For instance, such as the device 1400 shown in FIG. 14. As an example, the stimulator indicates a countdown to its next stimulation pulse followed by a second indication that it is sending the stimulus train. The automated detection device would then provide its own third audible indication of stimulation detection. In continuous monitoring uses, automated identification of the stimulation device 102 may be intended to improve both sensitivity (detect stimulation that might be obscured by, for example, respiration and EMG noise) and specificity (allow exclusion false electrical stimulation from, e.g. respiration, electrocautery).

In another aspect, the user interfaces 108 may allow for adjustment of an electrical stimulation pulse duration from a predefined range (e.g., about zero to about 200 microseconds). In one embodiment, the user interface 108 may be a potentiometer to allow a slide control to increase or decrease the electrical stimulation pulse duration within the predefined range. The stimulation pulse may have a non-adjustable frequency in the range of about 10 Hz to about 32 Hz, and desirably about 16 Hz or 32 Hz. In some embodiments, the stimulation pulse may comprise an adjustable frequency.

As a representative example, the stimulation pulse may have a biphasic waveform with controlled current during the cathodic (leading) phase, and net DC current less than 10 micro amps, switch adjustable from about 0.1 milliamps to about 20 milliamps, and pulse durations adjustable from about zero microseconds up to about 1 millisecond.

The operative element (e.g., probe 110) exits or attaches to the housing 120 at the nose cone 124 to deliver stimulus current to the excitable tissue. The probe 110 comprises a length and a diameter of a conductive material, and is desirably fully insulated with the exception of the uninsulated portion 114, e.g. about 1.0 millimeters to about 10 millimeters, and desirably about 4 millimeters to about 6 millimeters, which is non-insulated and serves as the stimulating to allow the surgeon to deliver the stimulus.

The size of the uninsulated portion 114, (the active electrode) of the probe 110 ensures a high current density that will stimulate nearby excitable tissue. The insulation portion 112 may comprise a medical grade heat shrink.

The conductive material of the probe 110 comprises a diameter having a range between about 0.5 millimeters to about 1.5 millimeters, and may be desirably about 1.0 millimeters. The length of the operative element 110 may be about 50 millimeters to about 60 millimeters, although it is to be appreciated that the length may vary depending on the particular application. As shown, the probe 110 may include one or more bends to facilitate accurate placement of the uninsulated portion 114. In one embodiment, the conductive material of probe 110 is made of a stainless steel, solid wire, although other conductive materials may be used. Further, the probe 110 may include an anchor 116. The anchor 116 may be of any appropriate configuration. By way of a non-limiting example, the anchor 116 may comprise a bend in the probe 110 such that upon insertion of the probe 110 into tissue of a patient, or more specifically, the anchor 116 being inserted into tissue of the patient, the anchor 116 prevents an undesired withdrawal of the anchor 116 and/or probe 110.

As previously described, in monopolar operation, a return electrode 131 (or indifferent electrode), for example, provides an electrical path from the body to the stimulation device 102. The return electrode 130 may be placed on the surface of intact skin (e.g., surface electrodes as used for electrocardiogic or electromyographic monitoring during surgical procedures) or it might be needle-like and be placed in the surgical field or penetrate through intact skin or an incision.

In some embodiments, the stimulating device 102 may include a probe/operative element 110 that is bipolar. The bipolar probe 110 may include a bipolar array of two contacts exposed on a distal end of the bipolar probe 110. The contacts may by way of example, have a diameter in the range of about 1-3 mm or potentially even less. The spacing between the contacts may be about 1 to 4 mm. Alternatively, a bipolar adapter may be utilized with the probe 110, such as disclosed in U.S. Pat. No. 10,154,792, which is incorporated herein by reference. In this embodiment, the burst mode (as described above) may enable a user to hold a consistent location/contact of the probe 110 with tissue but provide on/off contraction to assist with precise location or identification of a target nerve branch or fascicle.

The configuration of the stimulating medical devices that form a part of the system can vary in form and function. Various representative embodiments of illustrative medical devices will be described.

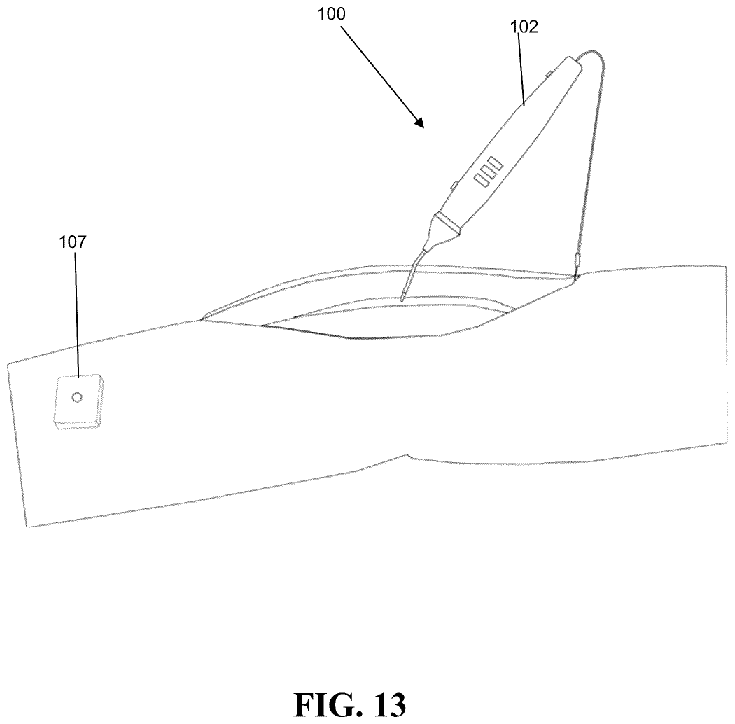

As shown in FIG. 13, the stimulation system 100 may be utilized during a surgical procedure to generate an electrical stimulation for the purposes of locating, stimulating, and evaluating nerve or muscle, or both nerve and muscle integrity. As shown, the stimulation device 102 may be held in the hand of the surgeon while performing the procedure. The evoked potential sensor 107 may be positioned on the patient in any applicable position, such as being spaced from the location of the surgical site where the nerve at issue innervates. However, it should be understood that the evoked potential sensor 107 may be positioned in more than the position shown. By way of a non-limiting example, it can be positioned at any location at which the nerve being stimulated innervates.

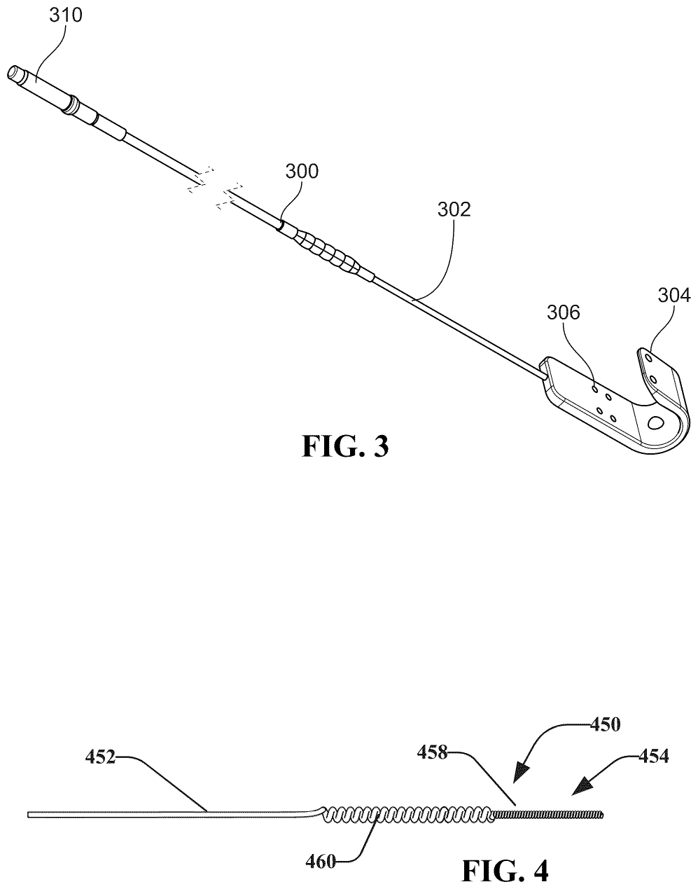

Referring now to FIGS. 3, this is percutaneous electrode 300 in accordance with various disclosed aspects. Percutaneous electrode 300 may generally include an insulated body 302, a nerve-engagement portion 304, and one or more electrodes 306. In an example, percutaneous electrode 300 may be placed at or near a target tissue region and may be coupled with a percutaneous lead or wire, as described herein. It is noted that percutaneous electrode 300 may be positioned while an incision is open and may be left in place while the incision is closed. In at least one other embodiment, percutaneous electrode 300 may be positioned when an incision is closed or by deploying the percutaneous electrode 300.

In embodiments, percutaneous electrode 300 may comprise strands of stainless steel wire or a single strand of wire insulated with a biocompatible polymer. Each wire strand may have a diameter of approximately 34 μm and the insulated multi-strand lead wire may have a diameter of approximately 250 μm. It should be understood, however, that these dimensions and materials are merely exemplary and the present teachings are not limited to such. Any appropriate sized, shaped and configured electrode and percutaneous lead may be used. The outer diameter of the percutaneous electrode 300 may be approximately 580 μm and it may be encased or filled with silicone or the like. In at least some embodiments, percutaneous electrode 300 may be made out of a different material (e.g., another metal, conducting polymer), may be insulated with another material, or may not be insulated. Further, the lead may be cylindrical or paddle-like.

Unlike surface electrodes that are applied to the surface of the patient's skin using an adhesive, percutaneous electrode 300 may be surgically implanted or otherwise inserted into select tissue. The nerve engagement portion 304 may comprise a generally J-shaped end that may be sized and shaped to be wrapped generally around the nerve or other location at which the electrical stimulation is to be applied. The J-shaped end 304 may be insulated or uninsulated or a portion thereof may be insulated and uninsulated. In at least some embodiments, the J-shaped end 304 may be inserted directly into tissue and may deliver electrical stimulation to the tissue. In another aspect, the J-shaped end 304 may act as an anchor by grabbing and holding on to the applicable nerve or other location at which electrical stimulation is applied. The J-shaped end may prevent the percutaneous electrode 300 from substantially moving or unintentionally coming loose. As shown in FIG. 3, disclosed embodiments may include different types of ends. For instance, an anchor may include tines. In an exemplary embodiment, a patch assembly may be utilized in conjunction with the percutaneous electrode 300. The patch assembly may comprise several layers, including an adhesive layer, an electrode layer, a reinforcement layer and a cover layer. In one embodiment, the patch assembly may include a power source for the stimulation device. Further, the patch assembly may act as a surface electrode. In one embodiment, the patch assembly may include an engagement member or members that electrically couple the stimulation device to the percutaneous electrode 300 to provide stimulation for nerve regeneration. The engagement member may comprise a snap, a magnetic male and female member capable of operable engagement, a bayonet engagement device, or any known engagement mechanisms capable of electrically coupling the stimulation device with the percutaneous electrode 300. The present disclosure contemplates any such configuration of the patch assembly.

In another aspect, an anchor may include threaded members (e.g., screws) or the like. Further still, the percutaneous electrode 300 may not include any tines or anchors. In these embodiments, the percutaneous electrode 300 may be placed near or around, i.e., generally circumscribing all of or a portion of the applicable nerve. Further, the percutaneous electrode 300 may be placed over, i.e., on top of or at the bottom of, the applicable nerve, or near, i.e., in an operative distance from the applicable nerve in any manner. The present teachings are not limited to a specific configuration. Embodiments may include a nerve cuff, a coiled lead, a straight lead, lead with a hook, lead with a tine or tines, or the like.

According to embodiments, percutaneous electrode 300 may comprise flexible materials that allow some or all of the percutaneous electrode 300 to bend or deform. In an example, the insulated portion 302 may be a lead that is generally flexible to allow removal, positioning, or other manipulation of the percutaneous electrode 300.

In embodiments, sections of the J-shaped end 304 may include one or more electrodes. In some embodiments, the J-shaped end 304 may include x number of electrodes, where x is a whole number between 1 and 10. The electrodes may operatively deliver a stimulation current. The percutaneous electrode 300 may include a quick connect end 310 that allows the percutaneous electrode to be operatively attached to a lead, adapter and/or may be operatively coupled with an electrical stimulation device.

FIG. 4 illustrates another percutaneous electrode 450 comprising an insulated body 452 and an anchor 454. The anchor 454 may comprise a twisted or braided wire. The anchor 454 may be electrically conducting and not insulated such that it may apply an electrical stimulation to tissue. In an aspect, the anchor 454 may comprise a helical portion 458. In another aspect, body 452 may include twisted, braided or helical portion 460. The helical portion 458 and helical portion 460 may anchor the percutaneous electrode 450 in places. Further, the helical portion 460 may comprise a fine-coiled wire with an insulative material surrounding such.

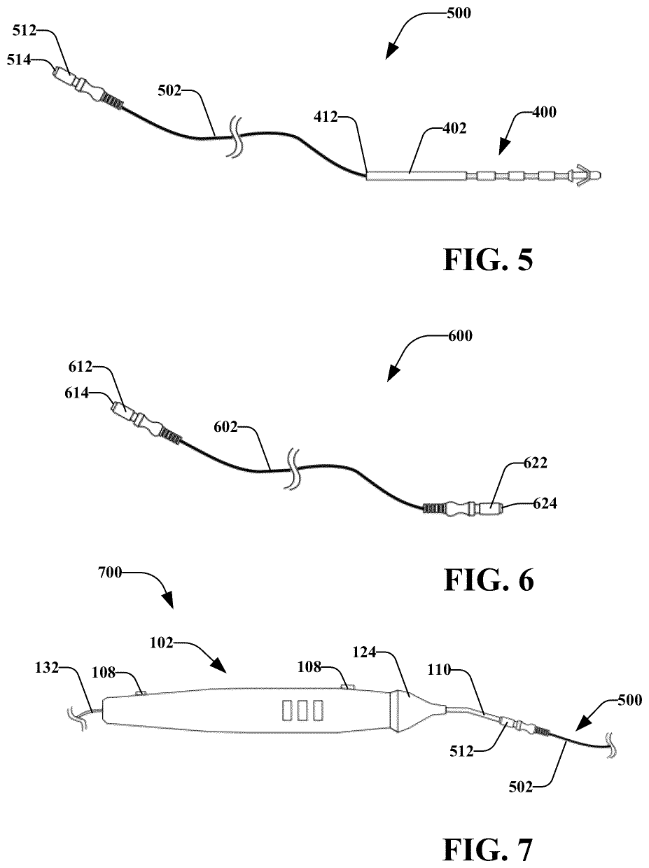

Turning now to FIGS. 5-7, with reference to FIGS. 1-4, there is adaptor 500 (which includes percutaneous electrode 400 (and may also include electrode 300 and 450) and a lead wire 502), adaptor 600 (which may be coupled to a stimulation device and/or percutaneous electrode), and stimulation system 700 (which may include stimulation device 102). It is noted that like named components of the various embodiments may comprise similar aspects, unless context suggests otherwise or a particular distinction is made.

In an embodiment, adaptor 500 may primarily include percutaneous electrode 400, wire 502 and connector 512. Wire 502 may connect terminal end 412 of the percutaneous electrode 400 with connector 512. In an aspect, wire 502 may comprise an insulated wire that is removably or irremovably attached to the percutaneous electrode 400 and/or connector 512. As described herein, the adaptor 500 may be configured to allow a stimulation probe 110 to deliver an electrical stimulation below the skin of a subject patient.

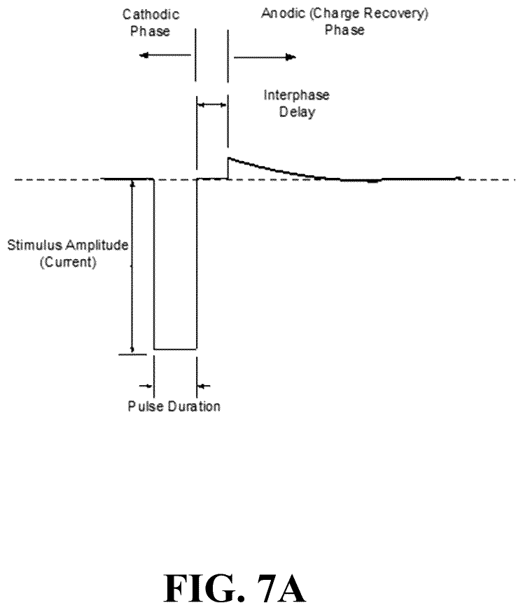

In an aspect, connector 512 may include an opening 514 that may receive an operative element. As shown in FIG. 7, the opening 514 may receive the probe 110 of a stimulation device 102. The opening 514 may be tapered to maintain the probe 110 in a friction tit within the connector 512. The connector may further include other retaining features, such as a fastener (e.g., screw, clasp, threaded portions VELCRO, magnet, etc.) to retain the connection between the connector 512 and the probe 110. It is noted that connector 512 may comprise an electrical connection disposed within the connector 512 that may operatively couple an uninsulated or stimulating portion of the probe 110 with the wire 502.

Wire 502 may extend from the connector 512. Wire 502 may be an electrical conductor in electrical connection with probe 110 when probe 110 is operatively inserted into the connector 512. It is noted that the wire 502 may be any appropriate length, such as 24 inches or a length between 12 inches and 48 inches. The lead wire may further be any appropriate gauge, such as 24 AWG wire.

Percutaneous electrode 400 may be coupled to the wire 502 at a terminal end 412. According to an embodiment, the wire 502 may be removably or irremovably coupled to the terminal end. It is noted that the wire 502 may be coupled directly to the terminal end 412 and/or may be coupled indirectly to the terminal end 412, such as through one or more other connectors (not shown). Moreover, wire 502 may be coupled to other portions of the percutaneous electrode 400. In an aspect, the connection between the wire 502 and the percutaneous electrode 400 may be insulated or uninsulated.

As shown in FIG. 6, adaptor 600 may comprise a wire 602 and one or more connectors 612/622. Each connector 612/622 may comprise an opening 614/624. In an aspect, openings 614/624 may comprise similar or different dimensions. For instance, openings 614/624 may be operatively sized and shaped to receive an operative element and/or a percutaneous electrode (e.g., percutaneous electrode 300/400). In at least one embodiment, opening 614 is operatively sized to receive an operative element, and opening 624 is operatively sized to receive a percutaneous electrode. In another aspect, connectors 612/622 may comprise elastomeric materials that may stretch, compress, or otherwise fit different sized components. Moreover, while embodiments disclose connectors 612/622 as female connectors, it is noted that one or more of connectors 612/622 may be a male connector. In another aspect, wire 602 (or 502) may comprise one or more branches or pathways such that connector 614, for example, may be electrically coupled with one or more other connectors through wire 602.

Turning to FIG. 7, and as described herein, system 700 may comprise stimulation device 102 that may be coupled with an adaptor 500 (or other described adaptors). In an aspect, a surgeon may utilize stimulation device 102 prior to or during a procedure (e.g., location of a nerve, nerve assessment, etc.). In some embodiments, the surgeon may place a percutaneous electrode or non-percutaneous electrode in a desired location prior to a surgical procedure. The surgeon may couple the electrode to the stimulation device 102 via connector 500. As illustrated, connector 512 may be attached to the probe 110. The surgeon may utilize user interfaces 108 to select a stimulation patterns.

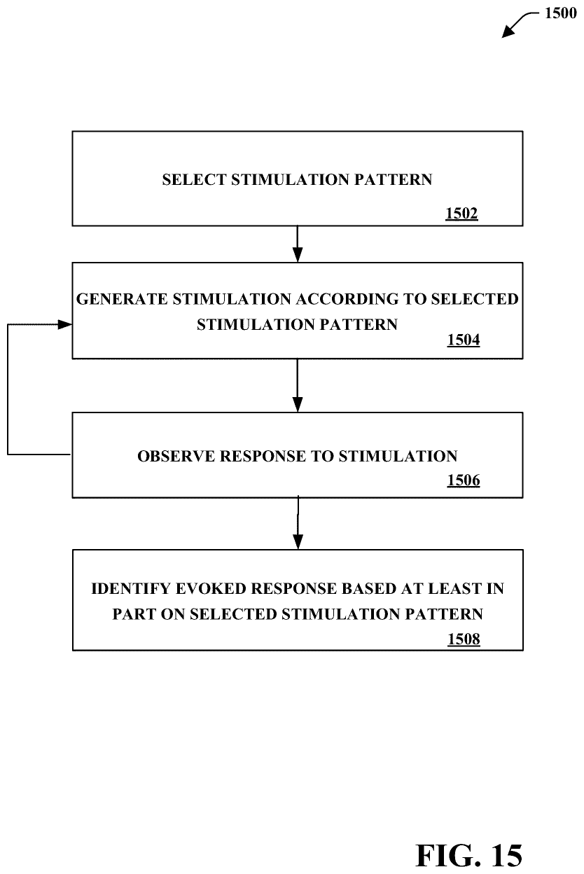

In view of the subject matter described herein, methods that may be related to various embodiments may be better appreciated with reference to the flowchart of FIG. 15. While the method is shown and described as a series of blocks, it is noted that associated methods or processes are not limited by the order of the blocks. It is further noted that some blocks and corresponding actions may occur in different orders or concurrently with other blocks. Moreover, different blocks or actions may be utilized to implement the methods described hereinafter. Various actions may be completed by one or more of users, mechanical machines, automated assembly machines (e.g., including one or more processors or computing devices), or the like.

FIG. 15 depicts an exemplary flowchart of non-limiting method 1500 associated with a stimulation system, according to various aspects of the subject disclosure. As an example, method 1500 may select stimulation patterns, generate stimulation and identify evoked responses to the stimulation patterns.

At 1502, a system may select a stimulation pattern for stimulation of target tissue. The selected pattern may include, for example, a burst or a wave pattern. It is noted that selection of a stimulation pattern may include interacting with an interface of a device such that the interface receives input or displays output.

At 1504, a system may generate stimulation according to the selected stimulation pattern. Generating the stimulation may include generating pulses at specified times and specified parameters.

At 1506, the system may observe response(s) to stimulation. For instance, the system may observe evoked potential, muscle twitches, or the like. The system may observe the stimulation via sensors or other devices. In some examples, a user my observe responses.

At 1508, the system may identify evoked response based at least in part on selected stimulation pattern. In an aspect, the responses may include slow-twitch muscle responses, patterns or time responses corresponding to the applied stimulation pattern, or the like. It is noted that filtration processes may remove inadvertent responses to improve identification of stimulation and nerve function.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. An electrical stimulation device comprising:
a housing;
an operative element extending from the housing;
a control circuitry in communication with the operative element, wherein the control circuit comprises a memory comprising instructions that when executed generate pulses of stimulus electrical stimulation that comprises i) at least first and second groups with a group time between a first and a last pulse of each of the first and second groups, and ii) a burst stimulation for a burst time between the first pulse of the first group and the first pulse of the second group, wherein the burst time is greater than the group time.

2. The electrical stimulation device of claim 1, wherein the burst stimulation comprises a frequency and amplitude to induce repeated slow-twitch contractions

3. The electrical stimulation device of claim 2, wherein the frequency is 1.6 Hz and a stimulus electrical frequency is 16 Hz.

4. The electrical stimulation device of claim 2, wherein the frequency is 2.7 Hz and a stimulus electrical frequency is 16 Hz.

5. The electrical stimulation device of claim 2, wherein the frequency is 3.2 Hz and a stimulus electrical frequency is 32 Hz.

6. The electrical stimulation device of claim 2, wherein the frequency is 4 Hz and a stimulus electrical frequency is 32 Hz.

7. The electrical stimulation device of claim 1 further comprising a user interface, wherein the control circuit modifies a pattern of the burst stimulation based on input from the user interface.

8. The electrical stimulation device of claim 1 further comprising a stimulation detection device configured to detect electrical stimulation to a target nerve.

9. The electrical stimulation device of claim 8, wherein the stimulation detection device comprises at least one of a return electrode, EMG sensor, a cuff, an endotracheal tube or a camera.

10. The electrical stimulation device of claim 1 further comprising an indicator configured to confirm delivery of a predefined stimulation pattern to tissue.

11. The electrical stimulation device of claim 1 further comprising a pulse counter configure to count a number of pulses applied.

12. The electrical stimulation device of claim 11, wherein the pulse counter is configured to turn off power when a predetermined number of pulses are detected by the pulse counter.

13. The electrical stimulation device of claim 1 further comprising an evoked potential sensor configured to determine generation of an evoked potential from a nerve being stimulated.

14. The electrical stimulation device of claim 13, wherein the evoked potential sensor comprises an accelerometer or an electrode.

15. An electrical stimulation device comprising:
a housing;
an operative element extending from the housing;
a control circuitry in communication with the operative element, wherein the control circuit comprises a memory comprising instructions that when executed generate pulses of stimulus electrical stimulation in a first group, wherein the pulses in the first group each comprise different parameters.

16. The electrical stimulation device of claim 15, wherein the first group comprises three pulses wherein each of the pulses comprise different amplitudes.

17. The electrical stimulation device of claim 15, wherein the first group comprises a plurality of pulses wherein each pulse comprises a different pulse duration.

18. An electrical stimulation device comprising:
a housing;
an operative element extending from the housing;
a control circuitry in communication with the operative element, wherein the control circuit generates pulses of electrical stimulation in a first group with a first group time between a first and a last pulse of the first group and a second group with a second group time between a first pulse and a last pulse and a burst stimulation for a burst time between the first pulse of the first group and the first pulse of the second group, wherein the burst time is greater than each of the first group time and the second group time.

19. The electrical stimulation device of claim 18, wherein the burst stimulation comprises a frequency and amplitude to induce repeated slow-twitch contractions.

20. The electrical stimulation device of claim 18, wherein the control circuit comprises a memory comprising third and fourth groups of pulses of electrical stimulation wherein stimulation parameters of the first and second groups are different from stimulation parameters of the third and fourth groups.

21. The electrical stimulation device of claim 20 further comprising an evoked potential sensor configured to determine generation of an evoked potential from a nerve being stimulated, wherein the evoked potential sensor changes the electrical stimulation from the first and second groups to the third and fourth groups.
